(19) United States
(12) Patent Application Publication
(10) Pub. No.: US 2024/0013910 A1
(43) Pub. Date: Jan. 11, 2024

(54) Method for Supporting a Patient's Health Control and Respective System

(71) Applicant: BIOTRONIK SE & Co. KG, Berlin (DE)

(72) Inventors: Shayan GUHANIYOGI, Portland, OR (US); Ravi Kiran Kondama REDDY, Portland, OR (US); R. Hollis WHITTINGTON, Portland, OR (US)

(73) Assignee: BIOTRONIK SE & Co. KG, Berlin (DE)

(21) Appl. No.: 18/253,760

(22) PCT Filed: Nov. 18, 2021
(86) PCT No.: PCT/EP2021/082156

§ 371 Date: May 19, 2023

Related U.S. Application Data
(60) Provisional application No. 63/118,170, filed on Nov. 25, 2020.

(30) Foreign Application Priority Data
Jan. 8, 2021 (EP) 21150731.4

(51) Int. Cl. G16H 40/67; G16H 50/20; G16H 50/30

(52) U.S. Cl. CPC G16H 40/67; G16H 50/20; G16H 50/30

(57) ABSTRACT

Method for supporting a patient's cardiac health control, using an at least partially extracorporeally located remote device comprising a processor and a memory connected to the processor, comprising the step of correcting those data value/values of at least one second data set of a physiological measure by means of a pre-defined correction factor determined for the respective second data set whose corresponding data value/values of a first data set of a physiological measure lies outside the first reference range by the processor, wherein the pre-defined correction factor of the respective second data set is previously determined for the respective second data set using a correction factor determining AI algorithm. The remote server is capable of analyzing the relationships among the collection of physiological data measured by the medical to reduce “information overload” and provide a more accurate picture of the patient's health condition.

### CROSS-REFERENCE TO RELATED APPLICATIONS

This application is the United States National Phase under 35 U.S.C. § 371 of PCT International Patent Application No. PCT/EP2021/082156, filed on Nov. 18, 2021, which claims the benefit of European Patent Application No. 21150731.4, filed on Jan. 8, 2021, and U.S. Provisional Patent Application No. 63/118,170, filed on Nov. 25, 2020, the disclosures of which are hereby incorporated by reference herein in their entireties.

### TECHNICAL FIELD

The present invention is directed to a system for supporting a patient's health control, in particular for the patient's health control, comprising an implantable medical device and a remote device. The remote device is located at least partially extracorporeally. The present invention is further directed to a respective remote device, a method for supporting the patient's health control, a respective computer program product and a respective computer readable data carrier.

### BACKGROUND

Active and passive implantable medical devices (IMDs), for example, a pacemaker (with leads), a BioMonitor (an implantable medical device recording heart rhythm data, in particular an electrocardiogram (ECG)), an Implantable Leadless Pacer (ILP), an Implantable Leadless Pressure Sensor (ILPS), an Implantable Cardiac Defibrillator (ICD) or a Shockbox (a Subcutaneously Implanted Cardiac Defibrillator (S-ICD)), contain sensors that collect physiological signals and transmit them as data to a physician device or to a remote server. The data collected from these various sensors can include, but are not limited to, ECG, impedance, activity, posture, heart sounds, blood pressure, respiration, and other data.

These data are measured in IMDs, the data are minimally processed on the IMD (including algorithms), and then the raw and/or minimally-processed and/or the results of the algorithm are transmitted to a remote device such as a physician device or a remote server where they can be viewed. Further long-term trends of each of these data are visualized to help guide patient care. The signals are generally processed and presented independently from the other numerous signals.

Processing the physiological data, or applying algorithms to the physiological data, exclusively on the IMD has drawbacks because it limits the extent of processing and performance of the algorithms due to the low-power requirements of IMDs. High-power, high-performance processing and algorithms cannot be performed on an IMD at the risk of sacrificing device longevity. Therefore, the clinical benefits of such processing and algorithms cannot be gained. Despite the use of minimal processing/algorithms on an IMD, these still have an impact on device longevity. In order to extend device longevity to provide longer clinical benefit, larger batteries must be used, thereby increasing the physical size of the IMD, which is typically undesired. As a result of the drawback above, increasing the size of the battery leaves less room for additional sensors which may provide even more physiologic data and clinical benefit.

Further, there are state-of-the-art solutions which generally treat each physiological data independently and present them to the end-user as such. Treating each physiological signal independently provides an inaccurate picture in certain cases. Further, presentation of numerous signals independently can result in “information overload” for the patient or health care provider (HCP), and may not provide a holistic and clear picture of the patient's health condition. As an example, the collection of physiological signals may be displayed as trends to the end-user, e.g., one trend for heart sounds amplitudes, another for DC impedance, another for posture, etc. Interpreting and using the numerous trends from all the physiological data (signals) to create an actionable health care plan for the patient can be difficult.

It is therefore desirable to receive a more accurate and faster generated picture of the health state of the patient which avoids information overload and, at the same time, does not have any impact on the longevity of the implantable medical device.

The above problem is solved by a method supporting a patient's health control with the features of claims 1 and 6, respectively, by a computer program product with the features of claim 11, by a computer readable data carrier with the features of claim 12, a remote device for supporting a patient's health control with the features of claims 13 and 14, respectively, and a respective system with the features of claim 15.

In particular, the problem is solved by a method for supporting a patient's health control, for example, the patient's cardiac health control, using and implanted medical device and an at least partially extracorporeally located remote device (i.e., the remote device is fully or partially located outside the patient's body) comprising a processor and a memory connected to the processor, wherein the method comprises the following steps:
receiving from the implanted medical device a first data set of data values of a first physiological measure by the remote device,
receiving from the implanted medical device at least one second data set of data values of a second physiological measure different from the first physiological measure by the remote device,
assessing each data item of the first data set by the processor whether its respective value lies within or outside a first reference range,
optionally assess each data item of one or more of the second data set whether its respective value lies within or outside a at least one second reference range by the processor
correcting those data value/values of the at least one second data set by means of a pre-defined correction factor determined for the respective second data set whose corresponding data value/values of the first data set lies outside the first reference range and/or the at least one second reference range by the processor and
storing the corrected data value/values of the at least one second data set with the respective second data set in the memory,
wherein the pre-defined correction factor of the respective second data set is previously determined for the respective second data set using a correction factor determining artificial intelligence (AI) algorithm.

After implantation the remote device receives raw data or minimally processed data of a first physiological measure and at least one second physiological measure from an implanted medical device. Physiological measure means any measurable aspect of a patient's physiology and includes digital and analog signals. Minimally processed data means processing technique supported by dedicated hardware (to avoid using software) of the medical device, for example, hardware filtering which is known filtering of the data done by module/circuit design of the electronic module of the medical device or other postprocessing technique (e.g., to minimize baseline drift of a signal using a high-pass filter). According to the present invention the data of the first physiological measure and the at least one second physiological measure are received, assessed and corrected in a remote device (rather than by the medical device) thereby increasing the longevity of the medical device if the battery size is kept constant or allowing to reduce the size of the medical device or to incorporate more sensors if the battery size is reduced.

Further, by using the remote device and an AI algorithm for determination of the correction factor the interdependency of data is taken into account which leads to more precise data. Additionally, the remote device is able to faster execute the highly sophisticated data analysis indicated above. According to the present invention, the advanced data processing and analysis will incorporate a collection of data together. For example, the inventors have recognized that many physiological data are influenced by a patient's posture (e.g., whether the patient is laying supine or their side), including ECG morphology, DC impedance, and heart sounds amplitudes. For example, QRS width, or QT interval, or heart sounds amplitudes without accounting for the patient's posture may provide an inaccurate picture of the patient's health condition, particularly if these measures are presented to the patient or an HCP in the form of trends.

The remote device provides corrected data values, for example, a corrected DC impedance value (value of the second data set) based on the patient's posture (data of the first data set). E.g., the DC impedance data of the patient measured during the time the patient was laying on their side are corrected as if they were detected while the patient is laying supine. Accordingly, the correction of the DC impedance values is only provided if the associated posture data value shows that the patient is not laying supine (i.e., is not within the first reference range) but, for example, on their side. While this example refers to a single signal (posture) to correct another single signal (DC impedance), in general any number of data may be used for any number of corrected measures. For example, a third data set may be corrected according to whether a first and second data set both lie outside of their respective reference ranges. This can be further extended to additional (e.g., fourth, fifth, etc.) data sets. Due to the complex nature of such interdependencies among various signals, the use of a correction factor determining AI algorithm is proposed. The correction factor is applied to those data values of the second data set which lie outside a first reference range. This first reference range is a data interval which includes the outer limits of the data interval. The first reference range may be pre-defined based on HCP experience or may be a result of the determination process of the correction factor using the AI algorithm. The first reference range defines the data value range of the first physiological measure for which the correction of the data value/values of the second physiological measure is not necessary.

Association of the data values among the multiple data sets are accomplished by storing certain time information with the data sets to allow reconstruction of timestamps for each data value in each data set. As an example, the medical device may store numerous data sets at the same time, and record the starting time of the storage and the sampling rates of each data set, thereby allowing reconstruction of timestamps for each data value within each data set by the remote device.

With regard to the present invention the processor is regarded as a functional unit of the remote device that interprets and executes instructions comprising an instruction control unit and an arithmetic and logic unit. The remote device is a computer, i.e., a functional unit that can perform substantial computations, including numerous arithmetic operations and logic operations without human intervention, such as, for example, a personal mobile device (PMD), a desktop computer, a server computer, clusters/warehouse scale computer or embedded system.

The above method is, for example, realized as a computer program which is a combination of above and below specified computer instructions and data definitions that enable computer hardware to perform computational or control functions or which is a syntactic unit that conforms to the rules of a particular programming language and that is composed of declarations and statements or instructions needed for a above and below specified function, task, or problem solution.

An algorithm is finite set of well-defined rules for the solution of the above problem in a finite number of steps or a sequence of operations for performing the above and below specific task.

The memory of the remote device may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device.

The present disclosure is directed toward overcoming one or more of the above-mentioned problems, though not necessarily limited to embodiments that do.

### SUMMARY

According to the present invention, the correction factor determining algorithm is an algorithm using methods of artificial intelligence. Artificial intelligence (AI) is a broad scientific discipline with its roots in philosophy, mathematics and computer science that aims to understand and develop systems that display properties of intelligence. AI algorithms comprises so-called machine learning algorithms where computers programs (algorithms) learn associations of predictive power from examples in data. Machine learning is most simply the application of statistical models to data using computers. Machine learning uses a broader set of statistical techniques than those typically used in medicine. AI algorithms further comprises so-called deep learning algorithms that are based on models with less assumptions about the underlying data and are therefore able to handle more complex data. Deep learning algorithms allow a computer to be fed with large quantities of raw data and to discover the representations necessary for detection or classification. Deep learning algorithms rely on multiple layers of representation of the data with successive transformations that amplify aspects of the input that are important for discrimination and suppress irrelevant variations. Deep learning may be supervised or unsupervised. AI algorithms further comprise supervised learning training computer algorithms to learn associations between inputs and outputs in data through analysis of outputs of interest defined by a (typically human) supervisor. Once associations have been learned based on existing data they can be used to predict future examples. AI algorithms further comprise unsupervised learning computer algorithms that learn associations in data without external definition of associations of interest. Unsupervised learning is able to identify previously undiscovered predictors, as opposed to simply relying on known associations. AI algorithms further comprise reinforcement learning computer algorithms that learn actions based on their ability to maximize a defined reward. This approach is influenced by behavioural psychology and has been applied with considerable success in gaming where there is perfect information, many possible options and no real world cost of failure. In one embodiment AI algorithms further comprise linear regression. In another embodiment AI algorithms further compromise classification techniques.

According to the present invention, those data value/values of the at least one second data set are corrected by means of a pre-defined correction factor determined for the respective second data set whose corresponding data value/values of the first data set lies outside the first reference range by the processor of the remote device, wherein the correction factor was previously determined for the respective second data set using the correction factor determining AI algorithm. “Previously determined” means that the determination of the correction factor is finished before it is used in the inventive method. However, the correction factor may be adapted occasionally using the correction factor determining AI algorithm, for example, in pre-defined time intervals. In one embodiment, those data value/values of the at least one second data set whose corresponding data value of the first data set lies within the first reference range are not changed.

In one embodiment the first physiological measure or the second physiological measure of the patient is, for example, one of the group comprising ECG signal data (e.g., heart rate, duration of P wave, duration of PR interval, duration of QRS complex, duration of QT interval, amplitude of the single heart beat), impedance, activity data, posture of the patient, diet data, heart sound, blood pressure, respiration signal data (e.g., respiratory rate, tidal volume, minute ventilation) and similar. However, the first physiological measure is different from the second physiological measure.

In one embodiment the pre-defined correction factor of the respective second data set is a single value and/or represents a mathematical function, wherein the single value is patient-specific or specific for a pre-determined group of patients, e.g., for patients of a specific age range and/or sex, or a general value for all patients, wherein the mathematical function is patient-specific or specific for a pre-determined group of patients, e.g., for patients of a specific age range and/or sex, or a general function for all patients. The mathematical function comprises a pre-defined mathematical dependency which was previously determined using the correction factor determining AI algorithm. The mathematical function is applied to the value of the second data set to be corrected. The single value is to be applied to the value of the second data set to be corrected using a pre-defined mathematical operation. The single value may replace the measured value of the second data set to be corrected.

In one embodiment, the pre-defined correction factor of the respective second data set is determined by training the correction factor determining AI algorithm using above mentioned AI training methods and data from a first learning period, e.g., one day, of the respective single patient or using data from a second learning period, e.g., one day or one week, of a group of at least two different patients, for example, a group of a plurality of patients. The length of the first and the second learning period may be similar or different, but each learning period may be chosen in a way that the determined correction factor is not affected by clinical changes.

In one embodiment the at least one second data set comprising the corrected data value/values may be displayed on a display unit of the remote device or on a display unit connected to the remote device in order to show the patient or the HCP the data of the second data set. The display unit may be formed by a computer monitor or screen having, for example, an electroluminescent (EL) display, a liquid crystal (LC) display, a light emitting diode (LED) display, an organic light emitting diode (OLED) display, an active matrix organic light emitting diode (AMOLED) display, a plasma (P) display or a quantum dot (QD) display. Those data are comparable with each other because they consider different condition of the patient (e.g., regarding their posture). The processor transmits the data sets to be displayed to the respective display unit. Accordingly, the display unit shows an accurate picture of the health condition of the patient.

The above problem is also solved in particular by a method for supporting a patient's health control, for example, the patient's cardiac health control, using an implanted medical device and an at least partially extracorporeally located remote device comprising a processor and a memory connected to the processor, wherein the method comprises the following steps:
receiving from the implanted medical device a first data set of data values of a first physiological measure covering at least a first time period by the implanted medical device,
receiving from the implanted medical device at least one second data set of data values of a second physiological measure covering at least the first time period by the remote device, wherein the second physiological measure is different from the first physiological measure,
assessing by the processor each data value of the first data set associated to the first time period and/or of the at least one second data set associated to the first time period and/or of at least one first trend value derived from the first data set for the first time period and/or of at least one second trend value derived from the at least one second data set associated to the first time period using a trend assessing AI algorithm thereby determining one first score value associated to the first time period from these data values and
storing the first score value associated to the first time period in the memory.

The terms used in this second method which correspond to the terms of the first inventive method explained above shall be analogously understood.

The above method is used to reduce “information overload” for the end-user such as a patient or an HCP. It was already explained above that interpreting and using the numerous trends from different physiological measures (see definition of physiological measure above), such as a first data set and a second data set, to create an actionable plan for patient is difficult. Hence, according to the present invention, the remote server uses a trend assessing AI algorithm to analyze the physiological data like the first data set and/or the at least one second data set and/or at least one trend value, for example, numerous trend values, derived from the first data set and/or the second data set and outputs a first score value related to the health condition of the patient. Preferably, the trend assessing AI algorithm uses a plurality of those data in order to determine the first score value in order to get a more accurate assessment. As an example, the algorithm may extract various features describing the trend from the data sets, including slopes, min/max, mean, etc. These features as well as the original first data set and the at least one second data set may then be input into a regression-based AI algorithm, such as a regression tree or an ensemble of regression trees. Briefly, a regression tree compares the input features to various thresholds in order to navigate through the tree, finally reaching the terminal node which assigns the final “score” based on the input features. In this case of regression, the final “score” relates to the patient's overall health.

Alternatively, the features as well as the original first data set and the at least one second data set may be input into a classification-based AI algorithm, such as an ensemble of classification trees. Briefly, a classification tree compares the input features to various thresholds in order to navigate through the tree, finally reaching the terminal node which assigns the final classification based on the input features. In the case of a single classification tree, the output may be a clinical outcome of interest, such as “healthy”, “pneumonia”, “heart failure decompensation”, etc. In the case of an ensemble of classification trees, the output of each tree is combined to generate a final “score” relating to the probability of each clinical outcome of interest. All data used by the AI algorithm in order to determine the first score value are associated to a pre-defined first time period in which these data were detected, for example, one particular hour or one particular day. Accordingly, the first trend value determined for a first time period is only one first score value associated with this time period that embodies all above mentioned data and characterizes the health condition of the patient, for example, their cardiac health condition for this time period. If this score value is observed over longer time, i.e., over a plurality of time periods, an overall trend may be determined from all scores from all covered time periods. For example, the determined score from each day observed over a full week or month may form another overall trend that the patient or HCP may view and use to inform clinical decision-making (instead of viewing the numerous individual trends for each physiological measure). Furthermore, the remote device may employ an additional higher-level algorithm that analyzes the score trend (or any other trend), and provides an alert when the trend matches a particular template or crosses a programmable threshold. This alert will further assist the HCP in their patient care.

Accordingly, as indicated above, the method may be executed for at least one second time period different from the first time period thereby determining one second score value for each second time period, wherein the at least one second score value is stored in the memory associated with the respective second time period. The first time period may be, for example, a first day and the second time period the following day. The first score and the numerous second scores form the above mentioned overall trend and may be analyzed using the above mentioned higher-level algorithm.

In one embodiment the data values of the first data set and/or the data values of the at least one second data set are corrected according to the method of claim 1.

In one embodiment the trend assessing AI algorithm comprises linear regression and/or regression using at least one regression tree, wherein the one first score value is selected from the at least one pre-defined regression tree and/or a pre-defined linear regression table for linear regression.

In one embodiment the pre-defined regression tree and/or pre-defined linear regression table is determined by training the trend assessing AI algorithm using above mentioned AI training methods and data from a third learning period, e.g., one day, of the respective single patient or using data from a fourth learning period, e.g., one day or one week, of a group of at least two different patients. The length of the first and the second learning period may be similar or different, but each learning period may be chosen in a way that the determined regression tree or linear regression table is not affected by clinical changes.

In one embodiment the first trend value derived from the first data set or the second trend value derived from the second data set may be one of a group comprising a slope of a pre-defined number of values of the first data set or the second data set, a minimum value of a pre-defined number of values of the first data set or the second data set, a maximum value of a pre-defined number of values of the first data set or the second data set, a mean value (e.g., arithmetic mean value, harmonic mean value, geometric mean value) of a pre-defined number of values of the first data set or the second data set, a median value of a pre-defined number of values of the first data set or the second data set and a standard deviation value of a pre-defined number of values of the first data set or the second data set. These trend values are useful trend values for health control which may be determined easily and with small effort.

As indicated above, the determined first score value and/or the at least one second score value may be used to automatically inform the patient or HCP about a pathological situation which may need further attention or in which the patient needs immediate help. Accordingly, these score values are compared with a pre-defined threshold value or a pre-defined template, wherein an electrical and/or audible and/or visible and/or tactile alarm signal is provided to a pre-defined person and/or device if the first score value and/or the at least one second score value are above or below the pre-defined threshold value and/or if the first score value and/or the at least one second score value do not match the pre-defined template. The use of a pre-defined template, or pattern, will allow detection of a more complex change in the patient's overall health compared to the use of pre-defined thresholds. An example, there may be a pre-defined “fluctuating” template which is defined as a pattern of periodically regular increases and decreases in the score values (e.g., a sinusoidal-type pattern). If the trend of score values matches this template pattern, a notification can be sent to the patient or HCP, as described above. In this particular example, the fluctuating nature of the patient's overall health (as described by the score values) can be informative to the HCP in guiding the patient's care to reduce such fluctuations and maintain the patient's overall health in a more consistent state.

In one embodiment, similar to the above data sets comprising corrected data values, the first score value and/or the at least one second score value may be displayed on a display unit of the remote device or on a display unit connected to the remote device. The processor transmits the score values to the respective display unit.

The present invention also comprises a combination of both above described methods. Accordingly, the data values of the first data set and/or the data values of the second data set are corrected according to the above method for data correction, preferably prior their assessment using the trend assessing AI algorithm. Similarly, the data values of the first data set and/or the data values of the second data set are corrected prior to determination of any first or second trend value. Accordingly, the score value is determined from corrected data and is therefore more reliable. Alternatively, instead of using a correction factor determined by an AI algorithm to consider the interdependencies among measures, and then a trend assessing AI algorithm to output a score based on the trends of these corrected physiological measures, there could be a single (combined) AI algorithm that does the interdependency correction and the output score in a single step. Such a combined AI algorithm may be trained by using a dataset containing many examples, each example having all the physiological measures of interest, and human annotations of each example. In the case of a regression-based AI algorithm, the human annotation for each example would be a score indicative of the overall health of the patient in that example. In the case of a classification-based AI algorithm, the human annotation for each example would be a label of the clinical outcome pertaining to that example. In either case, there would be a direct training of a final output score from the physiological measures. However, to use two AI algorithms consecutively, is desirable due to better interpretability for the user (less of a black box), and less potential for decreased performance (using several layers of independent AI algorithms has been shown to improve performance).

Another approach of such a combination, when using a classification-based AI algorithm, would be if the combined AI algorithm just reported the clinical outcome of interest instead of providing a score (in this case, the probability of that outcome) that could be interpreted by a trained user. As described above for the embodiment of a classification-based AI algorithm, the output score is related to the probability of a particular clinical outcome of interest. In this case, instead of providing this score (probability), the algorithm may simply report the clinical outcome that has the highest probability. For example, if the classification-based AI algorithm determines that the probability of a clinical outcome of “healthy” is 70%, and the probability of a clinical outcome of “pneumonia” is 30%, the algorithm will simply report “healthy”.

With the same advantages as explained above the problem is solved by a computer program product comprising instructions which, when executed by a processor, cause the processor to perform the steps of the methods indicated above. Similarly, the problem is also solved by a computer readable data carrier storing such computer program product.

Additionally, the above problem is solved with the same advantages as explained above by a remote device for supporting a patient's health control, for example, the patient's cardiac health control, wherein the remote device is at least partially extracorporeally located and comprises a processor and a memory connected to the processor, wherein the processor is configured to:
receive a first data set of data values of a first physiological measure,
receive at least one second data set of data values of a second physiological measure different from the first physiological measure,
assess each data item of the first data set whether its respective value lies within or outside a first reference range by the processor,
optionally assess each data item of one or more of the second data set whether its respective value lies within or outside a at least one second reference range by the processor
correct those data value/values of the at least one second data set by means of a pre-defined correction factor determined for the respective second data set whose corresponding data value/values of the first data set lies outside the first reference range and/or the at least one second reference range by the processor and
transmit the corrected data value/values of the at least one second data set to the memory in order to store the corrected data value/values of the at least one second data set with the respective second data set in the memory,
   wherein the remote device is further configured to previously determine the pre-defined correction factor of the respective second data set for the respective second data set using a correction factor determining AI algorithm. This remote device executes the above described method.

Similarly, the above problem is solved with the same advantages as explained above by a remote device for supporting a patient's health control, for example, the patient's cardiac health control, wherein the remote device is at least partially extracorporeally located and comprises a processor and a memory connected to the processor, wherein the processor is configured to:
receive a first data set of data values of a first physiological measure covering at least a first time period,
receive at least one second data set of data values of a second physiological measure covering at least the first time period, wherein the second physiological measure is different from the first physiological measure,
assess each data value of the first data set associated to the first time period and/or of the at least one second data set associated to the first time period and/or of at least one first trend value derived from the first data set for the first time period and/or of at least one second trend value derived from the at least one second data set associated to the first time period using a trend assessing AI algorithm and to determine thereby one first score value associated to the first time period from these data values and
transmit the determined first score value to the memory, wherein the memory is configured to store the first score value associated to the first time period.

The remote device may also execute a combination of above methods as described above. In particular, the processor of the device indicated in the previous paragraph is configured to correct data values of the first data set and/or the data values of the second data set according to the above described method, preferably prior their assessment using the trend assessing AI algorithm.

In one embodiment the remote device is further configured to:
correct data values of the first data set and/or the data values of the second data set according to the method of claim 1 prior their assessment using the trend assessing AI algorithm.

The problem is further solved by a system for supporting a patient's health control, for example, the patient's cardiac health control, comprising an implantable medical device and the remote device as described above, wherein the medical device comprises a sender for transmitting data of the first physiological measure (e.g., the first data set) and data of the at least one second physiological measure (e.g., the at least one second data set) to the remote device, wherein the remote device comprises a receiver or is connected to a receiver for receiving the data of the first physiological measure and the data of the at least one second physiological measure, wherein the receiver is connected to the processor. The receiver transmits the received data to the processor.

The implantable medical device may be a neurostimulator, a defibrillator, a pacemaker (with or without leads), or an implantable monitor.

The first data set and the at least one second data set may directly be transmitted from the medical device to the remote device by the sender wirelessly or by wire. For wireless transmission the sender may use radio wave communication, sonic communication (e.g., ultrasonic short range communication) or short range communication using electromagnetic induction. Wireless radio communication comprises, for example, WiFi communication, Bluetooth communication, NF communication. Accordingly, the sender of the medical device and the receiver of the remote device may be configured to execute those transmission methods. In one embodiment the data may be transmitted from the medical device to the remote device via a hub, for example, a patient's smart phone or another accessory device. This may occur by, for example, using Bluetooth, WiFi, or NF communication to transfer the data from the medical device to the hub, and then using any of the aforementioned wireless communication methods to transfer the data from the hub to the remote server.

The data of the first physiological measure and the data of the at least one second physiological measure may be transmitted in pre-defined time intervals (e.g., every night at a pre-defined time point, for example, at 3 a.m.). Alternatively or additionally, the data may be transmitted upon a respective request provided by the remote device. In this embodiment, the receiver is configured as transceiver and the data of the first physiological measure and the data of the at least one second physiological measure are transmitted by the sender from the medical device upon a respective request of the transceiver.

Units of the present disclosure such as the memory, a sensor or the processor may include any discrete and/or integrated electronic circuit components that implement analog and/or digital circuits capable of producing the functions attributed to the units herein. For example, the units may include analog circuits, e.g., amplification circuits, filtering circuits, and/or other signal conditioning circuits. The units may also include digital circuits, e.g., combinational or sequential logic circuits, memory devices, etc. The functions attributed to the units herein may be embodied as one or more processors, hardware, firmware, software, or any combination thereof. Depiction of different features as units is intended to highlight different functional aspects, and does not necessarily imply that such units must be realized by separate hardware or software components. Rather, functionality associated with one or more units may be performed by separate hardware or software components, or integrated within common or separate hardware or software components.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The present invention will now be described in further detail with reference to the accompanying schematic drawing, wherein

FIG. 1 shows a first embodiment of the inventive system comprising a medical device and a remote device, wherein the medical device is shown within a cross section of a patient's heart,

FIG. 2 depicts a flow chart of a first embodiment of an inventive method for supporting a patient's health control and

FIG. 3 depicts a flow chart of a second embodiment of an inventive method for supporting a patient's health control.

### DETAILED DESCRIPTION

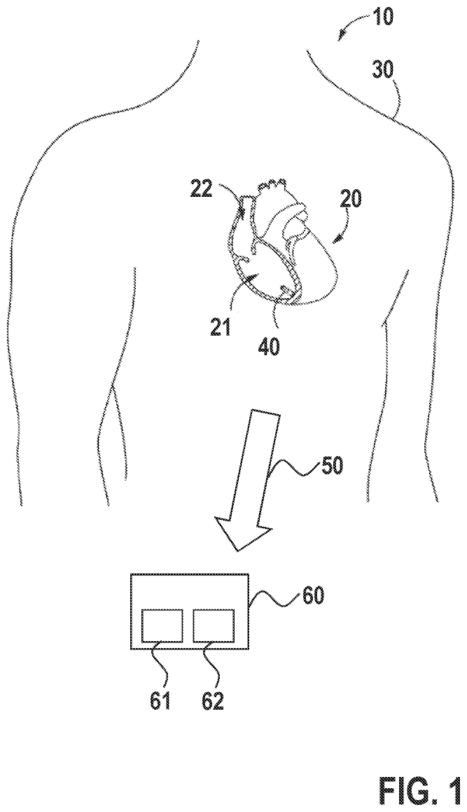

FIG. 1 shows an example medical system 10 and heart 20 of a patient 30. The system 10 comprises a leadless ventricular pacemaker device 40 (hereinafter “medical device 40”) and a remote device realized as remote server 60. Medical device 40 may be configured to be implanted within the right ventricle 21 of the heart and pace this ventricle, sense intrinsic ventricular depolarizations and impedance, and inhibit ventricular pacing in response to detected ventricular depolarization. The medical device may further comprise an accelerometer sensor in order to measure posture of the patient. A programmer (not shown) may be used to program medical device 40. A remote server 60 located outside the body of the patient may retrieve data of a first physiological measure and of at least one second physiological measure (e.g., posture data and impedance data) from medical device 40 at a pre-defined time point during night (e.g., at 3 a.m.) via Bluetooth communication (represented by arrow 50).

The medical device 40 may comprise a processing unit, a data memory, a signal generator unit for providing treatment signals (e.g., pacing signals), a measurement unit comprising an ECG measuring unit, an impedance sensor and the accelerometer sensor, a communication unit comprising a Bluetooth sender for transmitting the data of the first and the at least one second physiological measure as indicated above, and a power source wherein the units are electrically connected to each other. The power source may include a battery, e.g., a rechargeable or non-rechargeable battery. The data memory may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device and stores the measured data of the first physiological measure and the at least one second physiological measure.

The remote server 60 comprises a processor 61 and a memory 62 as defined above. For example, the processor may be a server-grade processor containing 56 cores to process large amounts of data, and the memory may a combination of RAM and NVRAM. The processor 61 implements a correction factor determining AI algorithm, for example, a linear regression.

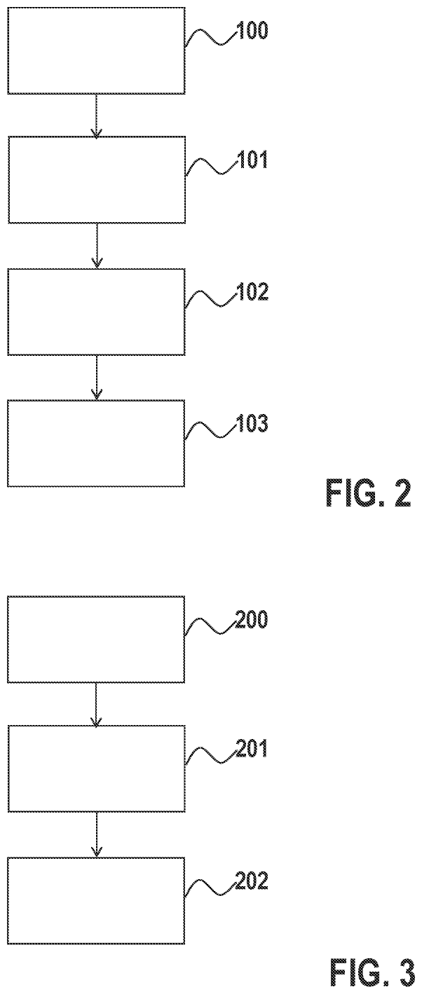

As an example, the remote server 60, in particular its processor 61, receives at the pre-defined time point every night (e.g., at 3 a.m.) a first data set of posture data values and a second data set of DC impedance data values from the medical device 40, wherein these data are measured for some duration (e.g., 1 minute) during the last 24 hours within the heart 20 of the patient 30 (step 100, see FIG. 2). In one embodiment, prior executing data correction using the correction factor the remote server 60 may use a learning period in which the posture and DC impedance data from one particular night (or multiple nights) are used to fits the relationship between DC impedance and posture for that patient in order to determine the correction factor. For example, during the course of one night, the DC impedance for the patient may be 1000 ohms when they are laying supine, but drops to 800 ohms when they turn to their side. The processor 61 collects this information and fits the relationship between the measured impedances and the postures during those measurements. The learning period should be short so that the fitted relationship is not affected by clinical changes.

The learning procedure of the correction factor determining AI algorithm is used to calculate the correction factor. As one example, the AI algorithm may be linear regression (which is covered by the correction factor determining AI algorithm) to determine the fitted relationships between measurements. In the example above, where the only interdependencies considered are DC impedance and posture, this is straightforward, but in examples where interdependencies are large (e.g., heart sounds amplitudes can be affected by posture, heart rate, activity, etc.), the use of AI is more critical. In such an example where interdependencies are large, the learning period should collect as many examples as possible, but still within a short period, such that the relationships are not affected by clinical changes. These examples are then fed into a linear regression AI algorithm (in this case, multiple linear regression due to the higher number of variables) to fit the relationships between the variables and thereby determine the correction factors.

In the straightforward DC impedance and posture example described above, the fitted relationship realized as correction factor may then be used to correct DC impedance data values provided by the medical device 40, wherein the correction factor may be a single value and/or represent a mathematical function which is to be applied to the DC impedance value to be corrected. In one example, the patient may spend one entire night sleeping on their back, and the next entire night sleeping on their side. If the medical device 40/remote server 60 were to report 1000 ohms as the DC impedance for the first night, and 800 ohms as the DC impedance for the second night, without taking into account the effects of posture, the user may believe there is a clinical change, when in fact there may not be.

Using the fitted relationship (i.e., the correction factor) from the learning period, the processor 61 of the remote server 60 corrects the DC impedance values for each day according to a reference posture (step 102 in FIG. 2). For that, the processor 61 of the remote server 60 previously analyses the posture data and determines using a pre-defined reference range whether the patient lies supine or at their side (see step 101 in FIG. 2). As an example, the reference posture might be the supine position (i.e., the reference range of posture data represents supine position of the patient), meaning that the remote server should report DC impedance values as would be expected when the patient is supine. This essentially determines the correction factor. In the example above, during the learning period it was detected that sleeping on the side for this patient resulted in a reduced impedance of 200 ohms compared to laying supine. Consequently, the correction for DC impedance measurements made when the patient is laying on their side (i.e., if the posture values are outside the reference range characterizing the supine position) is to add 200 ohms to the assigned measured DC impedance value. Alternatively, the correction may be a ratio (e.g., make a correction by adding 25% in this example). Finally, the DC impedance values, i.e., the corrected values and the values which did not need to be corrected, are stored in the memory 62 of the remote server (see step 103 in FIG. 2).

It is further emphasized that the reference for correction may not always be the physiological measure of posture. For example, the processor 61 may fit a relationship between heart sounds amplitudes and heart rate. In this case, the reference correction may be, for example, a 60 bpm heart rate. Another example could be a relationship between heart sounds amplitudes and activity level. In this case, the reference for correction may be an activity level of zero (no activity). It is possible that multiple references be taken into account for the corrected measure. For example, heart sounds amplitudes can be influenced by both heart rate and posture. In this case, the references could be both a 60 bpm heart rate and a supine posture. Making the correction for multiple references/interdependencies may be accomplished by the correction factor determining AI algorithm (as described above).

In another embodiment, instead of a learning period for each particular patient, the fitted relationships of the correction factor determining AI algorithm of the processor 61 may be derived from a training database (offline) of many patients.

As described above, the processor 61 may additionally or alternatively comprise a trend assessing AI algorithm that looks at the features of all the individual trends of physiological measures, and outputs a score based on these features. Hence, each day (or as frequently as the trends update), the trend assessing AI algorithm outputs a new score.

It is observed that the scores themselves determined over several days make up an overall (new) trend. Accordingly, the processor may comprise a further “higher level algorithm” which is an algorithm that looks at the trend of the determined scores over time. This higher level algorithm is not necessarily another AI algorithm, it can be a simple threshold-based algorithm (e.g., the score crosses a pre-defined threshold value), or a template-matching algorithm (e.g., the AI score trend matches a pre-defined template). The pre-defined threshold values or pre-defined templates that are clinically relevant may be determined using a database of many patient's trends.

When such a pre-programmed threshold is crossed or a predefined template is matched, the remote server will give an alert to the patient or HCP, for example, an electrical and/or audible and/or visible and/or tactile alarm signal. The electrical alarm signal may be conducted to another device which then provides a respective alarm, for example, to a respective monitoring authority. As one example, the pre-programmed threshold may be used as a general indicator of the patient's overall health. If the score increases above this threshold, then an alert is given to the patient. As another example, there may be a predefined template of the score trend that indicates the onset of a heart failure decompensation event. When the score trend matches this template, an alert is given to the patient.

Linear regression may be used as a trend assessing AI algorithm comprising a classic machine learning/AI technique in which generally a scalar (numerical) response is fitted against a number of explanatory variables. In the simple example referring to DC impedance and posture, the DC impedance may be the scalar response while posture is the explanatory variable. In a more complex example, the heart sounds amplitude may be the scalar response while the explanatory variables are heart rate, posture, and activity level. Using multiple data points (e.g., multiple measurements of DC impedance and the corresponding postures), the relationship between the scalar response and the explanatory variables can be calculated as weights/coefficients. In the case of the trend assessing AI algorithm, the scalar response is the output score, while the explanatory variables are characteristics of the trends of each physiological measure. Alternatively, a regression tree or an ensemble of regression trees may be used.

As one example of linear regression for the trend assessing AI algorithm, the explanatory variables may be chosen to be x1: the maximum of the DC impedance trend value, and x2: the average of the heart sounds amplitude trend value. x1 is calculated by taking all values stored in the DC impedance trend and using the processor 61 to apply a maximum function to these values. Similarly, x2 is calculated by taking all values stored in the heart sounds amplitude trend and using the processor 61 to apply the mean function to these values. After fitting the relationship between the output scores of the trend assessing AI algorithm and these two explanatory variables using a suitable training set, the trend assessing AI algorithm can then be used on the trends for each patient. For example, the trend assessing AI algorithm may provide an output score equal to 2*x1−0.5*x2.

As one example of a regression tree, the regression tree algorithm may determine that the score should be 5 if x1<500 ohms, 4 if x1>600 ohms AND x2<0.5\hat{ }⁻³ m/s², etc. Note that these output scores are examples only, and that the output scores could take on any range of values.

A regression tree used as the trend assessing AI algorithm allows fitting of more complex relationships between the explanatory variables and the scalar response. Instead of fitting a curve between the explanatory variables and the scalar response, it can break up the space of explanatory variables into decision regions and assign an expected scalar response to each region which forms the score. In another embodiment, an ensemble of regression trees forms the trend assessing AI algorithm that uses many such regression trees (each having different decision regions).

In another embodiment, an ensemble of classification trees forms the trend assessing AI algorithm. In this case, each classification tree outputs a label instead of a numerical value. As one such example, a classification tree algorithm may output a label of “pneumonia” 4 if x1<500 ohms, but may instead output a label of “healthy” if x1>600 ohms AND x2<0.5\hat{ }10⁻³ m/s², etc. All classification trees within the ensemble will have different decision criteria, and therefore will output a different label (in this example, “pneumonia” or “healthy”) depending on those criteria. Finally, the output labels of all such classification trees in the ensemble are used to determine the probability of the patient having “pneumonia” or being “healthy”.

Accordingly, the trend analysis according to the present invention comprises the steps receiving at the processor 61 a first data set of data values of a first physiological measure (e.g., posture values) covering at least a first time period (e.g., 1 specific day) and receiving at least one second data set of data values of a second physiological measure (e.g., DC impedance values) covering at least the first time period (e.g., the same specific day) from the medical device 40 (see step 200 of FIG. 3). Then, the processor 61 assesses, for example, each data value of posture data set, each data value of the DC impedance data set, the mean DC impedance value of that day and its standard deviation using the trend assessing AI algorithm, thereby determining one score value associated to this specific day (step 201 of FIG. 3). Then, the score value of this day is stored in the memory 62 (step 202 of FIG. 3). For the data of the next day, a new score is determined and stored analogously. As indicated above, the score value may be compared with a pre-defined threshold value by the processor 61 in order to determine whether the patient faces a pathological situation. If the determined score value is greater than a pre-determined threshold value, an audible alarm signal is provided to the patient by the remote server 60.

The advantage of the trend analysis using the trend assessing AI algorithm is that it combines many physiological parameters, rather than one, which gives a more comprehensive picture of the patient's health condition, and which may give new indications, which could be overseen or not recognized by the (human) user because of the big amount of data.

In a further embodiment, the above explained data correction method and the above explained determination of score value for trend assessing may be combined as explained above. Alternatively, instead of using a first AI algorithm to correct the interdependencies among measures, and then a second AI algorithm to output a score based on the trends of these corrected measures, there could be a single AI algorithm that does the interdependency correction and the output score in a single step.

The corrected data of the at least one second physiological measure (DC impedance values) may be displayed on a respective display unit of the remote server 60 with an LCD screen. Additionally or alternatively, the determined score values may be displayed at the same screen.

The technical advantage of this present invention is the ability to implement high-performance processing and AI analyses at a server-level in order to extend the longevity of the medical device 40 without increasing battery size and/or decreasing the size of the medical device 40 and/or incorporating more physiologic sensors into the medical device 40 to provide more clinical benefit to the patient 30. Further, the inventive remote server 60 is capable of analyzing the relationships among the collection of physiological data measured by the medical to reduce “information overload” and provide a more accurate picture of the patient's health condition.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

The present invention further comprises the embodiments indicated by the following numbered examples:

1. A method for supporting a patient's health control, for example, the patient's cardiac health control, using an at least partially extracorporeally located remote device comprising a processor and a memory connected to the processor, wherein the method comprises the following steps:
receiving a first data set of data values of a first physiological measure,
receiving at least one second data set of data values of a second physiological measure different from the first physiological measure,
assessing each data item of the first data set by the processor whether its respective value lies within or outside a first reference range,
optionally assess each data item of one or more of the second data set whether its respective value lies within or outside a at least one second reference range by the processor
correcting those data value/values of the at least one second data set by means of a pre-defined correction factor determined for the respective second data set whose corresponding data value/values of the first data set lies outside the first reference range and/or the at least one second reference range by the processor and
storing the corrected data value/values of the at least one second data set with the respective second data set in the memory,
wherein the pre-defined correction factor of the respective second data set is previously determined for the respective second data set using a correction factor determining AI algorithm.

2. The method of example 1, wherein those data value/values of the at least one second data set whose corresponding data value of the first data set lies within the first reference range are not changed.

3. The method of any of the previous examples, wherein the correction factor determining AI algorithm is a linear regression or a deep learning algorithm.

4. The method of any of the previous examples, wherein the pre-defined correction factor of the respective second data set is a single value and/or represents a mathematical function, wherein the single value is patient-specific or specific for a pre-determined group of patients or a general value for all patients, wherein the mathematical function is patient-specific or specific for a pre-determined group of patients or a general function for all patients.

5. The method of any of the previous examples, wherein the pre-defined correction factor of the respective second data set is determined by training the correction factor determining AI algorithm using data from a first learning period of the respective single patient or using data from a second learning period of a group of at least two different patients.

6. The method of any of the previous examples, wherein first physiological measure or the second physiological measure is one of a group comprising ECG signal data (e.g., heart rate, duration of P wave, duration of PR interval, duration of QRS complex, duration of QT interval, amplitude of the single heart beat), impedance, activity data, posture of the patient, diet data, heart sound, pressure, respiration signal data (e.g., respiratory rate, tidal volume, minute ventilation) or similar.

7. The method of any of the previous examples, wherein the at least one second data set comprising the corrected data value/values is displayed on a display unit of the remote device or on a display unit connected to the remote device.

8. A method for supporting a patient's health control, for example, the patient's cardiac health control, using an at least partially extracorporeally located remote device comprising a processor and a memory connected to the processor, wherein the method comprises the following steps:
receiving a first data set of data values of a first physiological measure covering at least a first time period,
receiving at least one second data set of data values of a second physiological measure covering at least the first time period, wherein the second physiological measure is different from the first physiological measure,
assessing by the processor each data value of the first data set associated to the first time period and/or of the at least one second data set associated to the first time period and/or of at least one first trend value derived from the first data set for the first time period and/or of at least one second trend value derived from the at least one second data set associated to the first time period using a trend assessing AI algorithm thereby determining one first score value and/or one first label associated to the first time period from these data values and
storing the first score value and/or one first label associated to the first time period in the memory.

9. The method of example 8, wherein the trend assessing AI algorithm comprises
a) linear regression and/or regression using at least one regression tree, wherein the one first score value is selected from the at least one pre-defined regression tree and/or a pre-defined linear regression table for linear regression and/or
b) classification using at least one classification tree, wherein the one first label is selected by comparing the at least one first trend values and/or at least one second trend values with at least one threshold.

10. The method of any of examples 8 to 9, wherein the method is executed for at least one second time period different from the first time period thereby determining one second score value and/or second label for each second time period, wherein the at least one second score value and/or second label is stored in the memory associated with the respective second time period.

11. The method of any of examples 8 to 10, wherein the pre-defined regression tree and/or pre-defined linear regression table and/or classification tree is determined by training the trend assessing AI algorithm using data from a third learning period of the respective single patient or using data from a fourth learning period of a group of at least two different patients.

12. The method of any of the examples 8 to 11, wherein the first physiological measure or the second physiological measure is one of ECG signal data (e.g., heart rate, duration of P wave, duration of PR interval, duration of QRS complex, duration of QT interval, the amplitude of the single heart beat), impedance, activity data, posture of the patient, diet data, heart sound, blood pressure, body weight, oxygen saturation, respiration signal data (e.g., respiratory rate, tidal volume, minute ventilation) or similar.

13. The method of any of the examples 8 to 12, wherein the first trend value derived from the first data set or the second trend value derived from the second data set is one of a group comprising a slope of a pre-defined number of values of the first data set or the second data set, a minimum value of a pre-defined number of values of the first data set or the second data set, a maximum value of a pre-defined number of values of the first data set or the second data set, a mean value (e.g., arithmetic mean value, harmonic mean value, geometric mean value) of a pre-defined number of values of the first data set or the second data set, a median value of a pre-defined number of values of the first data set or the second data set and a standard deviation value of a pre-defined number of values of the first data set or the second data set.

14. The method of any of the examples 8 to 13, wherein the first score value and/or the at least one second score value are compared with a pre-defined threshold value or a pre-defined template, wherein an electrical and/or audible and/or visible and/or tactile alarm signal is provided to a pre-defined person and/or device if the first score value and/or the at least one second score value are above or below the pre-defined threshold value and/or if the first score value and/or the at least one second score value do not match the pre-defined template.

15. The method of any of the examples 8 to 14, wherein the first score value and/or the at least one second score value are displayed on a display unit of the remote device or on a display unit connected to the remote device.

16. The method of any of the examples 8 to 15, wherein the data values of the first data set and/or the data values of the second data set are corrected according to the method of any of the examples 1 to 7, preferably prior their assessment according to examples 8 to 15.

17. Computer program product comprising instructions which, when executed by a processor, cause the processor to perform the steps of the method according to any of the examples 1 to 16.

18. Computer readable data carrier storing a computer program product according to example 17.

19. A remote device for supporting a patient's health control, for example, the patient's cardiac health control, wherein the remote device is at least partially extracorporeally located and comprises a processor and a memory connected to the processor, wherein the processor is configured to:
receive a first data set of data values of a first physiological measure,
receive at least one second data set of data values of a second physiological measure different from the first physiological measure,
assess each data item of the first data set whether its respective value lies within or outside a first reference range by the processor,
optionally assess each data item of one or more of the second data set whether its respective value lies within or outside a at least one second reference range by the processor,
correct those data value/values of the at least one second data set by means of a pre-defined correction factor determined for the respective second data set whose corresponding data value/values of the first data set lies outside the first reference range and/or the at least one second reference range by the processor and
transmit the corrected data value/values of the at least one second data set to the memory in order to store the corrected data value/values of the at least one second data set with the respective second data set in the memory,
wherein the remote device is further configured to previously determine the pre-defined correction factor of the respective second data set for the respective second data set using a correction factor determining AI algorithm.

20. The device of example 19, wherein those data value/values of the at least one second data set whose corresponding data value of the first data set lies within the first reference range are not changed.

21. The device of any of the examples 19 to 20, wherein the correction factor determining AI algorithm is a linear regression or a deep learning algorithm.

22. The device of any of the examples 19 to 21, wherein the pre-defined correction factor of the respective second data set is a single value and/or represents a mathematical function, wherein the single value is patient-specific or specific for a pre-determined group of patients or a general value for all patients, wherein the mathematical function is patient-specific or specific for a pre-determined group of patients or a general function for all patients.

23. The device of any of the examples 19 to 22, wherein processor is further configured to determine the pre-defined correction factor of the respective second data set by training the correction factor determining AI algorithm using data from a first learning period of the respective single patient or using data from a second learning period of a group of at least two different patients.

24. The device of any of the examples 19 to 23, wherein first physiological measure or the second physiological measure is one of ECG signal data (e.g., heart rate, duration of P wave, duration of PR interval, duration of QRS complex, duration of QT interval, amplitude of the single heart beat), impedance, activity data, posture of the patient, diet data, heart sound, pressure, respiration signal data (e.g., respiratory rate, tidal volume, minute ventilation) or similar.

25. The device of any of the examples 19 to 20, wherein the remote device comprises a display unit connected to the processor and the memory, wherein the display unit is configured to display the at least one second data set comprising the corrected data value/values.

26. A remote device for supporting a patient's health control, for example, the patient's cardiac health control, wherein the remote device is at least partially extracorporeally located and comprises a processor and a memory connected to the processor, wherein the processor is configured to:
receive a first data set of data values of a first physiological measure covering at least a first time period,
receive at least one second data set of data values of a second physiological measure covering at least the first time period, wherein the second physiological measure is different from the first physiological measure,
assess each data value of the first data set associated to the first time period and/or of the at least one second data set associated to the first time period and/or of at least one first trend value derived from the first data set for the first time period and/or of at least one second trend value derived from the at least one second data set associated to the first time period using a trend assessing AI algorithm and to determine thereby one first score value and/or one first label associated to the first time period from these data values and
transmit the determined first score value to the memory, wherein the memory is configured to store the first score value and/or one first label associated to the first time period.

27. The device of example 26, wherein the trend assessing AI algorithm comprises
a) linear regression and/or regression using at least one regression tree, wherein the one first score value is selected from the at least one pre-defined regression tree and/or a pre-defined linear regression table for linear regression and/or
b) classification using at least one classification tree, wherein the one first label is selected by comparing the at least one first trend values and/or at least one second trend values with at least one threshold.

28. The device of any of the examples 26 to 27, wherein the processor is configured to execute the steps for at least one second time period different from the first time period thereby determining one second score value and/or second label for each second time period and to transmit the at least one second score value and/or second label associated with the respective second time period to the memory for storing it in the memory.

29. The device of any of the examples 26 to 28, wherein the pre-defined regression tree and/or pre-defined linear regression table and/or classification tree is determined by training the trend assessing AI algorithm using data from a third learning period of the respective single patient or using data from a fourth learning period of a group of at least two different patients.

30. The device of any of the examples 26 to 29, wherein first physiological measure or the second physiological measure is one of ECG signal data (e.g., heart rate, duration of P wave, duration of PR interval, duration of QRS complex, duration of QT interval, the amplitude of the single heart beat), impedance, activity data, posture of the patient, diet data, heart sound, pressure, respiration signal data (e.g., respiratory rate, tidal volume, minute ventilation) or similar.

31. The device of any of the examples 26 to 30, wherein the first trend value derived from the first data set or the second trend value derived from the second data set is one of a group comprising a slope of a pre-defined number of values of the first data set or the second data set, a minimum value of a pre-defined number of values of the first data set or the second data set, a maximum value of a pre-defined number of values of the first data set or the second data set, a mean value (e.g., arithmetic mean value, harmonic mean value, geometric mean value) of a pre-defined number of values of the first data set or the second data set, a median value of a pre-defined number of values of the first data set or the second data set and a standard deviation value of a pre-defined number of values of the first data set or the second data set.

32. The device of any of the examples 26 to 31, wherein the processor is configured to compare the first score value and/or the at least one second score value with a pre-defined threshold value or a pre-defined template, wherein an alarm signal is generated by the processor and a signal generator of the device is configured to send the electrical and/or audible and/or visible and/or tactile alarm signal to a pre-defined person and/or device if the first score value and/or the at least one second score value are above or below the pre-defined threshold value and/or if the first score value and/or the at least one second score value do not match the pre-defined template.

33. The device of any of the examples 26 to 32, wherein the remote device comprises a display unit connected to the processor and the memory, wherein the display unit is configured to display the first score value and/or the at least one second score value.

34. The device of any of the examples 26 to 33, wherein the processor is configured to correct data values of the first data set and/or the data values of the second data set according to the method of any of the examples 1 to 7, preferably prior their assessment using the trend assessing AI algorithm.

35. A system for supporting a patient's health control, for example, the patient's cardiac health control, comprising an implantable medical device and the remote device according to any of the examples 19 to 34, wherein the medical device comprises a sender for transmitting data of the first physiological measure and data of the at least one second physiological measure to the remote device, wherein the remote device comprises a receiver or is connected to a receiver for receiving the data of the first physiological measure and the data of the at least one second physiological measure, wherein the receiver is connected to the processor.

36. The system of example 35, wherein the data of the first physiological measure and the data of the at least one second physiological measure are transmitted by the sender wirelessly or by wire.

37. The system of any of the examples 35 to 36, wherein the receiver is configured as transceiver and the data of the first physiological measure and the data of the at least one second physiological measure are transmitted upon a respective request of the transceiver.

1. A method for supporting a patient's health control, for example the patient's cardiac health control, using an implanted medical device and an at least partially extracorporeally located remote device comprising a processor and a memory connected to the processor, wherein the method comprises the following steps:
receiving from the implanted medical device a first data set of data values of a first physiological measure by the remote device,
receiving from the implanted medical device at least one second data set of data values of a second physiological measure different from the first physiological measure by the remote device,
assessing each data item of the first data set by the processor whether its respective value lies within or outside a first reference range,
correcting those data value/values of the at least one second data set by means of a pre-defined correction factor determined for the respective second data set whose corresponding data value/values of the first data set lies outside the first reference range by the processor, and
storing the corrected data value/values of the at least one second data set with the respective second data set in the memory,
wherein the pre-defined correction factor of the respective second data set is previously determined for the respective second data set using a correction factor determining an AI algorithm.

2. The method of claim 1, wherein the correction factor determining the AI algorithm is a linear regression or a deep learning algorithm.

3. The method of claim 1, wherein the pre-defined correction factor of the respective second data set is a single value and/or represents a mathematical function, wherein the single value is patient-specific or specific for a pre-determined group of patients or a general value for all patients, wherein the mathematical function is patient-specific or specific for a pre-determined group of patients or a general function for all patients.

4. The method of claim 1, wherein the pre-defined correction factor of the respective second data set is determined by training the correction factor determining the AI algorithm using data from a first learning period of the respective single patient or using data from a second learning period of a group of at least two different patients.

5. The method of claim 1, wherein the at least one second data set comprising the corrected data value/values is displayed on a display unit of the remote device or on a display unit connected to the remote device.

6. A method for supporting a patient's health control, for example the patient's cardiac health control, using an implanted medical device and an at least partially extracorporeally located remote device comprising a processor and a memory connected to the processor, wherein the method comprises the following steps:
receiving from the implanted medical device a first data set of data values of a first physiological measure covering at least a first time period by the remote device,
receiving from the implanted medical device at least one second data set of data values of a second physiological measure covering at least the first time period by the remote device, wherein the second physiological measure is different from the first physiological measure,
assessing by the processor at least one first trend value derived from the first data set for the first time period and/or of at least one second trend value derived from the at least one second data set associated to the first time period using a trend assessing an AI algorithm thereby determining one first score value and/or one first label associated to the first time period from these data values, and
storing the first score value and/or one first label associated to the first time period in the memory.

7. The method of claim 6, wherein the trend assessing the AI algorithm comprises
a) linear regression and/or regression using at least one regression tree, wherein the one first score value is selected from the at least one pre-defined regression tree and/or a pre-defined linear regression table for linear regression, and/or
b) classification using at least one classification tree, wherein the one first label is selected by comparing the at least one first trend values and/or at least one second trend values with at least one threshold.

8. The method of any of claim 6, wherein the pre-defined regression tree and/or pre-defined linear regression table and/or classification tree is determined by training the trend assessing the AI algorithm using data from a third learning period of the respective single patient or using data from a fourth learning period of a group of at least two different patients.

9. The method of claim 6, wherein the first score value and/or the at least one second score value are compared with a pre-defined threshold value or a pre-defined template, wherein an electrical and/or audible and/or visible and/or tactile alarm signal is provided to a pre-defined person and/or device if the first score value and/or the at least one second score value are above or below the pre-defined threshold value and/or if the first score value and/or the at least one second score value do not match the pre-defined template.

10. The method of claim 6, wherein the data values of the first data set and/or the data values of the second data set are corrected prior their assessment.

11. Computer program product comprising instructions which, when executed by a processor, cause the processor to perform the steps of the method according to claim 1.

12. Computer readable data carrier storing a computer program product according to claim 11.

13. A remote device for supporting a patient's health control, for example the patient's cardiac health control, wherein the remote device is at least partially extracorporeally located and comprises a processor and a memory connected to the processor,
wherein the processor is configured to:
receive a first data set of data values of a first physiological measure,
receive at least one second data set of data values of a second physiological measure different from the first physiological measure,
assess each data item of the first data set whether its respective value lies within or outside a first reference range by the processor,
correct those data value/values of the at least one second data set by means of a pre-defined correction factor determined for the respective second data set whose corresponding data value/values of the first data set lies outside the first reference range by the processor, and
transmit the corrected data value/values of the at least one second data set to the memory in order to store the corrected data value/values of the at least one second data set with the respective second data set in the memory,
wherein the remote device is further configured to previously determine the pre-defined correction factor of the respective second data set for the respective second data set using a correction factor determining an AI algorithm.

14. A remote device for supporting a patient's health control, for example the patient's cardiac health control, wherein the remote device is at least partially extracorporeally located and comprises a processor and a memory connected to the processor,
wherein the processor is configured to:
receive a first data set of data values of a first physiological measure covering at least a first time period,
receive at least one second data set of data values of a second physiological measure covering at least the first time period, wherein the second physiological measure is different from the first physiological measure,
assess at least one first trend value derived from the first data set for the first time period and/or of at least one second trend value derived from the at least one second data set associated to the first time period using a trend assessing AI algorithm and to determine thereby one first score value and/or one first label associated to the first time period from these data values, and
transmit the determined first score value to the memory, wherein the memory is configured to store the first score value and/or one first label associated to the first time period.

15. A system for supporting a patient's health control, for example the patient's cardiac health control, comprising an implantable medical device and the remote device according to claim 13, wherein the medical device comprises a sender for transmitting data of the first physiological measure and data of the at least one second physiological measure to the remote device, wherein the remote device comprises a receiver or is connected to a receiver for receiving the data of the first physiological measure and the data of the at least one second physiological measure, wherein the receiver is connected to the processor.
